(19) United States
(12) Patent Application Publication
(10) Pub. No.: US 2026/0076826 A1
(43) Pub. Date: Mar. 19, 2026

(54) SURGICAL INSTRUMENTS FOR PHACOEMULSIFICATION SURGERY

(71) Applicant: Johnson & Johnson Surgical Vision, Inc., Irvine, CA (US)

(72) Inventor: Chetan Nirkhe, Aliso Viejo, CA (US)

(21) Appl. No.: 18/888,585

(22) Filed: Sep. 18, 2024

(51) Int. Cl. A61F 9/007; A61M 1/00

(52) U.S. Cl. CPC A61F 9/00745; A61M 1/774; A61M 2210/0612

(57) ABSTRACT

A surgical instrument for phacoemulsification procedures is disclosed. The surgical instrument may include a handpiece assembly and a cleaning member. The cleaning member may be coupled with the handpiece assembly. The cleaning member may be configured to be inserted into anterior chamber of the eye of a patient and into a capsular bag of the eye. The cleaning member may include one or more polishing elements configured to detach material from the capsular bag.

### FIELD

The present disclosure generally relates to the field of ophthalmology, and more specifically, to surgical instruments for use in phacoemulsification procedures, such as cataract surgery.

### BACKGROUND

Certain surgeries, such as phacoemulsification surgery, have been successfully employed in the treatment of various ocular problems, such as cataracts. Phacoemulsification surgery is performed when cataracts cause a person's eye lens to become cloudy. The lens is one of the parts of the eye that is responsible for focusing light necessary to create clear images of objects at various distances. The lens is located inside a capsular bag, which is behind the iris and the cornea.

During phacoemulsification surgery, a small incision is made in the cornea of an eye and a tip of a phacoemulsification handheld surgical instrument, or handpiece, is inserted through the corneal incision and into the interior of the capsular bag. The handpiece includes a needle which is ultrasonically driven once placed within the capsular bag to break up and emulsify the lens and/or the cataract into small pieces. The emulsified lens may subsequently be removed using the same handpiece, or a different handpiece, in a controlled manner. For example, the emulsified lens may be aspirated from the capsular bag using a handpiece with an aspiration tip (e.g., needle) that is similar to the ultrasound phacoemulsification tip, but without the ultrasonic vibration.

The aspiration tip may also be used to rub against the internal surfaces of the capsular bag to help detach and remove any remaining cortical material (e.g., residual cortical fibers or epithelium cells) that remain attached or stuck to the capsular bag. The process of detaching and/or removing the residual cortical material from the capsular bag is referred to as “polishing” the capsular bag. The capsular bag should be completely polished to detach and remove the residual cortical material in order to prevent the formation of another cataract.

However, the aspiration tip of the handpiece may not be able to detach all of the residual cortical material from the capsular bag. For example, some cortical material attached to the capsular bag may be resistant to aspiration. As a result, the residual cortical material may remain adhered to the capsular bag. Further, the aspiration process may be time consuming and the movement of the aspiration tip over the capsular bag may be limited or restricted due to the handpiece design and/or configuration. Thus, a surgeon may not be able to access all regions or areas of the capsular bag, including the sub-incisional cortex, to clean and/or aspirate the residual cortical material from the capsular bag. In addition, conventional aspiration tips are typically made from titanium or stainless steel and may contain burrs or sharp edges that may increase the risk of tearing a hole in the capsular bag. Damage to the capsular bag may limit the type of intraocular lens that can be used, impair effective healing, and/or prevent satisfactory visual recovery. For at least the foregoing reasons, there is a need for a device or instrument for polishing a capsular bag following cataract extraction to detach and remover material from the capsular bag.

### BRIEF DESCRIPTION OF THE DRAWINGS

FIG. 1 is a front view of a surgical instrument, in accordance with one example;

FIG. 2 illustrates a cleaning member attached to the tip of the surgical instrument of FIG. 1;

FIG. 3 is a front perspective view of a cleaning member configured to be attached to the surgical instrument of FIG. 1;

FIG. 4 is a perspective view of a surgical instrument, in accordance with another example;

FIG. 5A is a top view of a cleaning member of the surgical instrument of FIG. 4;

FIG. 5B is a side view of the cleaning member of FIG. 5A; and

FIG. 6A is a side view of a cleaning member of a surgical instrument, in accordance with another example;

FIG. 6B is a side view of the cleaning member of FIG. 6A;

FIG. 7A is a top view of a cleaning member of a surgical instrument, in accordance with another example;

FIG. 7B is a side view of the cleaning member of FIG. 7A; and

FIG. 8 is a partial perspective view of a surgical instrument, in accordance with another example.

### DESCRIPTION

Before explaining the examples in detail, it should be noted that the present disclosure is not limited in its application or use to the details of construction and arrangement of parts illustrated in the accompanying drawings and description, because the illustrative examples may be implemented or incorporated in other examples, variations and modifications, and may be practiced or carried out in various ways. Furthermore, unless otherwise indicated, the terms and expressions employed herein have been chosen for the purpose of describing the examples of the present disclosure for the convenience of the reader and are not for the purpose of limitation.

The present disclosure is directed to surgical instruments for use in phacoemulsification procedures, such as the removal of cataracts from eyes of patients. The surgical instruments are lightweight, easy to use, relatively inexpensive to manufacture, and reduce the risk of damaging a capsular bag of an eye. The surgical instruments enable a surgeon to more quickly, effectively, and safely polish the capsular bag (e.g., clean, wipe, scrape, rub, etc.) to detach and remove any remaining cataract material (e.g., cortical material) from the capsular bag following phacoemulsification. The remaining emulsified lens and/or cataract tissue may be detached more efficiently and effectively than traditional techniques.

Further, the surgical instruments can be manipulated by a surgeon to access and reach all regions of the interior of the capsular bag. For example, the surgical instruments may enable the surgeon to completely polish the capsular bag including the sub-incisional cortex. The surgical instruments may include a cleaning member having one or more polishing elements. The polishing elements may include one or more wiping edges and/or one or more polishing surfaces. The cleaning member may be constructed of a relatively soft material to reduce the risk of damaging the capsular bag.

In one aspect, a surgical instrument for phacoemulsification procedures is disclosed. The surgical instrument may include a handpiece assembly and a cleaning member. The cleaning member may be coupled with the handpiece assembly. The cleaning member may be configured to be inserted into anterior chamber of the eye of a patient and into a capsular bag of the eye. The cleaning member may include one or more polishing elements configured to detach material from the capsular bag.

In another aspect, a method of polishing a capsular bag of an eye of a patient is disclosed. The method may comprise performing a phacoemulsification procedure on an eye of a patient. The method may comprise inserting a surgical instrument having a cleaning member into the capsular bag of the patient. The cleaning member may include one or more polishing elements configured to detach material from the capsular bag. Further, the method may comprise moving the cleaning member to cause the one or more polishing elements to detach the material from the capsular bag. In addition, the method may comprise removing the surgical instrument from the capsular bag.

In use, a surgeon may insert the cleaning member of a surgical instrument into an incision in the cornea of a patient's eye and into the capsular bag after a cataract has been removed. For example, the surgical instrument may be inserted into an anterior chamber of the eye through an incision and into the capsular bag. The surgeon can scrape, wipe, clean, and detach remaining material (e.g., emulsified lens and cataract material, cortical material, etc.) from the surfaces of the capsular bag using one or more of the polishing elements of the cleaning member. The design of the surgical instrument enables the surgeon to use the cleaning member to effectively and completely polish the capsular bag including the sub-incisional cortex. In some examples, the cleaning member may not be a separate component that is attached to a handpiece assembly, but instead may be integrated or combined with the handpiece assembly to form a unitary or single piece surgical polishing instrument, which may be achieved using techniques known in the art, including, but not limited to a machining technique or some other similar method.

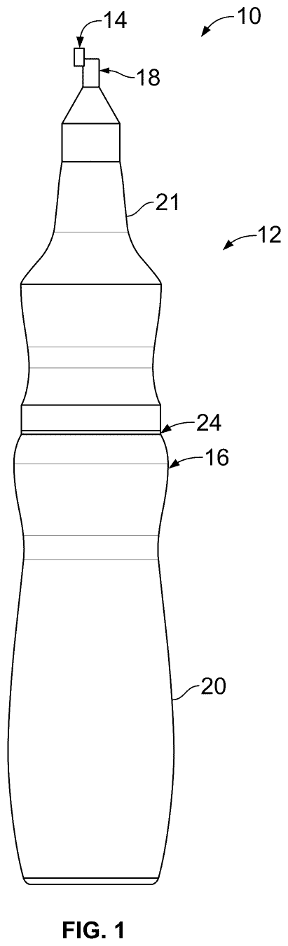

Referring now to the drawings, and particularly to FIG. 1, an example of a surgical instrument 10 is illustrated. The surgical instrument 10 generally includes a handpiece assembly 12 and a cleaning member 14. As illustrated, the handpiece assembly 12 includes an elongated body 16 and a tip 18. The elongated body 16 of the surgical instrument 10 may be adapted to be gripped and held by a surgeon or user. As a result, the surgical instrument 10 may be easily manipulated by the surgeon to treat an eye of a patient. For example, the surgeon may use the surgical instrument 10 to scrape, wipe, clean, and/or polish the capsular bag of the patient's eye.

The elongated body 16 of the surgical instrument 10 may have a substantially circular cross-sectional shape of various widths, but may have any suitable shape and/or size which allows it to be grasped by the surgeon. some examples, the elongated body 16 may be textured along its length to provide a surgeon with a suitable gripping surface. In other examples, the elongated body 16 may include anti-slip material to facilitate gripping the elongated body 16 by the surgeon. The elongated body 16 of the surgical instrument 10 may be constructed or manufactured from a variety of synthetic resin materials including plastic (i.e., Delrin, polyethylene, ABS, nylon, or polycarbonate), titanium, aluminum, steel, or any other suitable material.

As shown in FIG. 1, the elongated body 16 of the surgical instrument 10 includes a handle 20 and a grip portion 21. The grip portion 21 of the surgical instrument 10 may be coupled or attached with the handle 20. The grip portion 21 may be disposable to eliminate re-sterilization of the portions of the grip portion 21 that enter into the patient's eye. As such, the grip portion 21 may be detached and/or removed from the handle 20 after use and disposed of. The grip portion 21 may be detachably coupled to the handle 20 mechanically, such as by a threaded connection (e.g., locking threads), an interlocking mechanism, magnetic contacts, an adhesive, or any other suitable connection. As illustrated, the handle 20 may be threadedly attached at a connecting junction 24 to the grip portion 21. This design allows a surgeon to attach different griping portions to the handle 20.

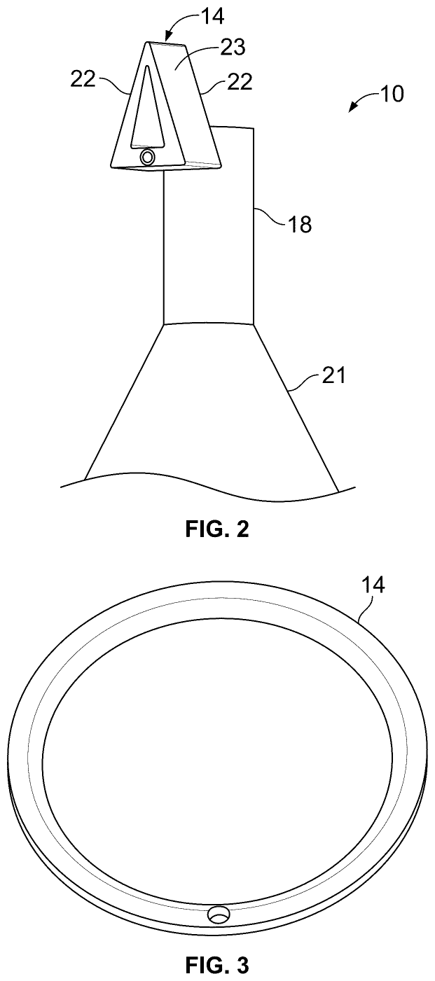

As shown in FIG. 2, the cleaning member 14 of the surgical instrument may be coupled with the tip 18 of the surgical instrument 10. The cleaning member 14 may be connected or coupled to the cleaning member 112 to tip 18 by any suitable means. Although the cleaning member 14 is shown as a separate component, it will be recognized that the cleaning member 14 may be integrated or combined with the grip portion 21 of the handpiece assembly 12.

The cleaning member 14 is sized and shaped to fit through an incision made in the cornea of an eye of the patient and to engage with the interior surfaces of a capsular bag. For example, the surgical instrument 10 may be inserted into an anterior chamber of the eye through an incision and into the capsular bag. As illustrated, the cleaning member 14 may have a generally triangular shape. In other examples, the shape of the cleaning member 14 may be circular, semi-circular, elliptical, parabolic, rectangular, curved, curvilinear, arced, etc. FIG. 3 shows another example of a cleaning member 14 for attachment to the surgical instrument of FIG. 1 having a circular shape.

The configuration of the cleaning member 14 enables a surgeon to manipulate the cleaning member 14 to a desired position in the patient's eye to polish (by scraping, wiping, cleaning, etc.) the capsular bag to detach and/or remove cortical material from the interior surfaces of the capsular bag such as the sub-incisional area. The cleaning member 14 may be formed from any suitable material to enable a surgeon to detach material (e.g., emulsified lens and cataract material, cortical material, etc.) from the capsular bag of a patient's eye to polish the capsular bag. The cleaning member 14 may be configured to provide a cushioning and/or non-abrasive engagement with the capsular bag. For example, the cleaning member 14 may be constructed from a soft polymeric segment or material or any other suitable material. In other examples, the cleaning member 14 may be formed from silicone, polyurethane, polyethylene, polypropylene, polystyrene, polytetrafluoroethylene, rubber, latex, or other medically compatible polymers or plastic compounds.

As shown in FIG. 2, the cleaning member 14 of the surgical instrument 10 may include one or more polishing elements. The polishing elements of the cleaning member 14 are configured to polish (by wiping, scrapping, and/or cleaning) the capsular bag to physically abrade cells or cortical material from the surfaces of the capsular bag. For example, the polishing elements may be used to detach remaining strands of cortex or cells still attached to the capsular bag and to polish the capsular bag. The polishing elements of the cleaning member 14 may be formed from a soft polymeric segment or any other material capable of polishing the capsular bag.

The polishing elements of the surgical instrument may include one or more wiping edges 22 and/or one or more polishing surfaces 23. In some examples, at least one of the wiping edges 22 may be part of a wiper member or blade. The polishing surfaces 23 may be substantially smooth or may have any other suitable shape or configuration. In some examples, the cleaning member 14 may also include bristles, brushes, textured materials, or the like to improve cleaning and/or polishing of the capsular bag.

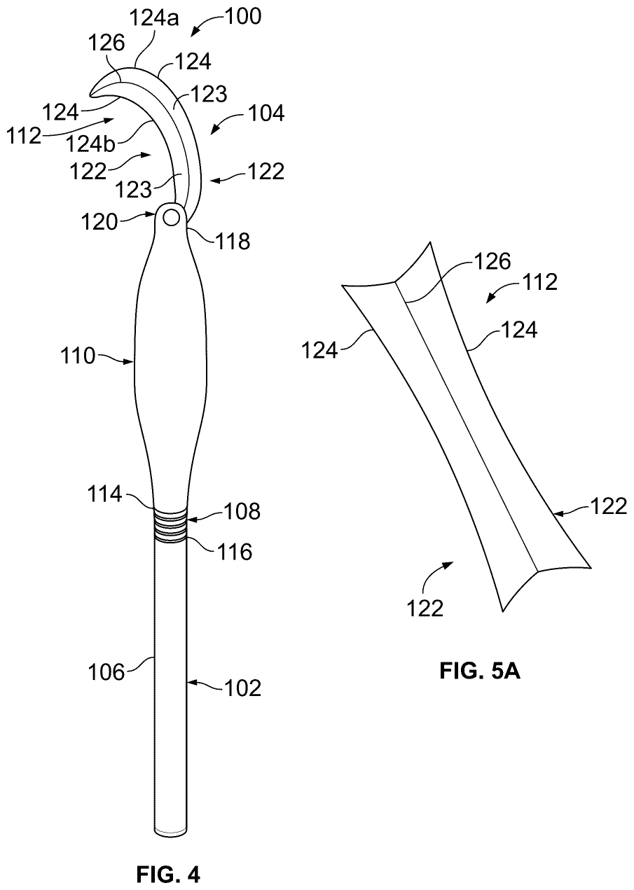

Referring now to FIG. 4, a surgical instrument 100, in accordance with another example, is illustrated. The surgical instrument 100 generally includes a handpiece assembly 102 and a surgical tool 104. The handpiece assembly 102 may comprise a handle 106. The handpiece assembly 102 may be a relatively thin, linear, and elongated member with opposing ends. The handpiece assembly 102 may have a substantially circular cross-sectional shape and may be adapted to be gripped and held by a surgeon or user, but may be any suitable shape or size which allows it to be grasped by the surgeon. In some examples, the handpiece assembly 102 may be textured along its length to provide a surgeon with a suitable gripping surface. In other examples, the handpiece assembly 102 may include anti-slip material to facilitate gripping the handpiece assembly 102 by the surgeon. The handpiece assembly 102 of the surgical instrument 100 may be constructed or manufactured from a variety of materials including plastic (i.e., polyethylene, nylon, or polycarbonate or a combination of soft and hard segments of synthetic resin), titanium, aluminum, steel, or any other suitable material. While a single or unitary handpiece assembly 102 is illustrated, the handpiece assembly may comprise multiple parts or pieces.

The handpiece assembly 102 of the surgical instrument 100 may be coupled with or attached to the surgical tool 104. The surgical tool 104 may be disposable to eliminate re-sterilization of the portions of the surgical instrument 100 that enter the patient's eye. As such, the surgical tool 104 may be detached or removed from the handpiece assembly 102 after use and disposed of. The surgical tool 104 may be detachably coupled with the handpiece assembly 102 by a threaded connection, interlocking mechanism, or any other suitable connection. As illustrated, the handpiece assembly 102 is threadedly attached at a connecting junction 108 to the surgical tool 104. This design allows a surgeon to attach different surgical tools to the handpiece assembly 102. The location of the connecting junction 108 may be anywhere along the handpiece assembly 102.

The surgical tool 104 of the surgical instrument 100 includes a shaft 110 and a cleaning member 112 (e.g., a tip portion). As shown in FIG. 4, the shaft 110 is generally linear and elongated with a first end 114 and an opposing second end 118. The shaft 110 may be disposed between the handpiece assembly 102 and the cleaning member 112. The first end 114 of shaft 110 is coupled with a distal portion 116 of the handpiece assembly 102 and the second end 118 of the shaft 110 is coupled with cleaning member 112. In some examples, the shaft 110 may be configured at different angles relative to the handpiece assembly 102.

The shaft 110 of the surgical tool 104 may have a substantially circular cross-sectional shape and may be constructed or manufactured from a synthetic resin such as plastic material (e.g., polyethylene) or any other suitable material (e.g., metal, etc.). The shaft 110 may have any suitable length to spatially separate the handpiece assembly 102 from the cleaning member 112 so that a surgeon's hand is at a suitable distance from the patient's eye. The shaft 110 may also have any suitable shape and/or thickness. As a result, the surgical instrument 100 may be easily manipulated by the surgeon to treat (e.g., scrape, wipe, clean, and/or polish) the capsular bag of the patient's eye.

A connecting member 120 may connect or couple the cleaning member 112 with the shaft 110. In some examples, the connecting member 120 may enable the cleaning member 112 to be articulated or pivotally moveable relative to the shaft 110 and/or rotatable about an axis of the shaft 110 to position the cleaning member 112 at a desired position for treating an eye of a patient. Thus, a surgeon may position the cleaning member 112 at a preselected angle and/or orientation relative to the shaft 110.

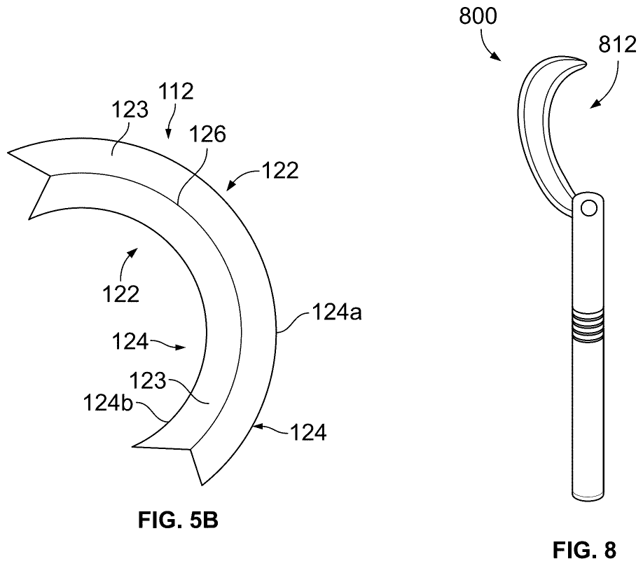

The cleaning member 112 of the surgical tool 104 may be sized and shaped to fit through an incision made in the cornea or sclera of an eye of the patient and to engage with the interior surfaces of the capsular bag. FIG. 5A shows a top view of the cleaning member 112, and FIG. 5B shows a side view of the cleaning member 112. As illustrated, the cleaning member 112 may have a generally curved or arc shape. In other examples, the shape of the cleaning member 112 may be semi-circular, elliptical, parabolic, rectangular, triangular, arched, or any other suitable shape or configuration to enable cortical material to be detached and removed from the surface of the capsular bag while maintaining the integrity of the bag.

The cleaning member 112 may be formed from any suitable material to enable a surgeon to wipe, scrape, clean, and/or detach material from the capsular bag of a patient's eye and to polish the capsular bag. The cleaning member 112 may be textured to enable easier detachment of the material from the capsular bag, but may be designed/shaped to prevent capsular tears or other damage to the capsular bag. In one example, the cleaning member 112 may be constructed from a soft polymeric segment or material but any other suitable material (or a combination of material), including a polymer or other similar material and/or a metal or other similar material, may be used. The polymeric portion/material may be attached at any location on the cleaning member 112 when more than one material is used. In some examples, the cleaning member 112 may be formed from silicone, polyurethane, polyethylene, polypropylene, polystyrene, POM/Delrin, polytetrafluoroethylene, rubber, latex, or other medically compatible polymers or plastic compounds.

As shown in FIG. 4, the cleaning member 112 of the surgical tool 104 may include one or more polishing elements 122. The one or more polishing elements 122 may be configured to clean and/or polish the capsular bag. The polishing elements 122 may be used to physically abrade cells or cortical material from the surfaces of the capsular bag. For example, the polishing elements 122 may be used to detach remaining strands of cortex or cells still attached to the capsular bag after cataract removal and to polish the capsular bag.

The polishing elements of the cleaning member 112 may include one or more polishing surfaces 123 and/or one or more wiping edges 124 for detaching and removing material from the surface of the capsular bag. The polishing surfaces 123 and the wiping edges 124 may be used to detach and remove material from the capsular bag and to clean and/or polish the capsular bag. The polishing surfaces 123 may be substantially flat, smooth, rounded, curved, or any other suitable shape and may be fabricated from a soft polymetric material or other suitable material to reduce the risk of damaging the capsular bag. The polishing surfaces 123 may be formed from plastic, composite material, or other suitable materials capable of polishing a capsular bag. As such, the capsular bag can be polished with the cleaning member 112 of the surgical instrument 100 to detach and remove any attached cells or material from the capsular bag. In some examples, the polishing surfaces 123 may include a textured portion or material to help detach material from the capsular bag. In some examples, the cleaning member 112 may also include one or more bristles, brushes, abrasive materials, etc. to clean and/or polish the capsular bag.

As shown in FIGS. 5A and 5B, the cleaning member 112 may include a groove 126 defined between a first edge 124a and a second edge 124b of the wiping edges 124 of the cleaning member 112. The first and second edges 124a and 124b of the cleaning member 112 may be used for wiping surfaces of the capsular bag to detach and remove material, such as cortical material, from the capsular bag. When the capsular bag is scraped with the first and/or second edges 124a and 124b of the cleaning member 112, material may be collected in the groove 126. The groove 126 may be formed in the top surface of the cleaning member 112 between the first and second edges 124a and 124b. In some examples, the groove 126 may be formed at or near the middle of the top surface. The groove 126 may define an angle between the first and second edges 124a and 124b. The angle can be between 10 degrees and 60 degrees, but any suitable angle may be formed. In some examples, the bottom of the groove 126 may lie in a different plane than the first and second edges 124a and 124b which may lie in the same plane or different planes.

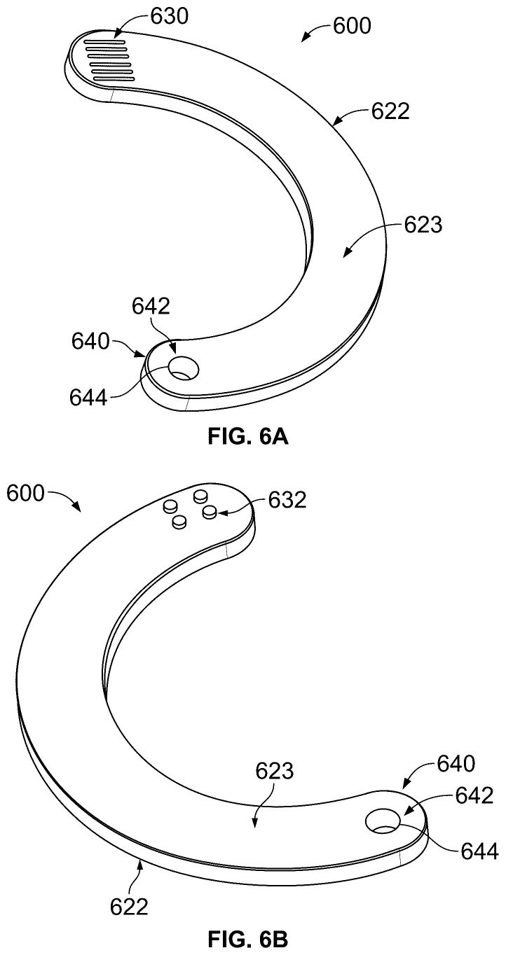

FIGS. 6A and 6B show another example of a cleaning member 600 for attachment to a handle or handpiece of a surgical instrument. For example, the cleaning member 600 may be configured to be coupled with the shaft 110 of the surgical instrument 100 of FIG. 4. The cleaning member 600 may be coupled with the handle or handpiece by any suitable means and in any suitable configuration. The cleaning member 600 may have an attachment portion 640. The attachment portion 640 may be coupled with or attached to a handle or handpiece of a surgical instrument. The attachment portion 640 may be defined at any suitable location of the cleaning member 600. A connection mechanism 642 may be used to couple the cleaning member 600 with a handle or handpiece of a surgical instrument. As shown, the connection mechanism 642 may comprising a through-hole 644 for receiving a connecting member (not shown), but may comprise any suitable mechanism for coupling the cleaning member 600 with a surgical instrument. Although the cleaning member 600 is shown as a separate component, it will be recognized that the cleaning member 600 may be integrated or combined with a handle or handpiece assembly to form a unitary or single piece surgical polishing instrument.

The cleaning member 600 may be used by a surgeon to polish a capsular bag of an eye. For example, the cleaning member 600 may be configured to detach and remove material (e.g., cortical material) from the interior surfaces of the capsular bag by wiping, scraping, detaching, etc. the interior surfaces of the capsular bag. As illustrated, the cleaning member 600 may have a generally curved or arced shape. The cleaning member 600 may be constructed from a soft polymeric segment or any other suitable material.

As shown FIGS. 6A and 6B, the cleaning member 600 may include one or more polishing elements. The polishing elements may be configured to polish the capsular bag. The polishing elements may include one or more wiping edges 622 and one or more polishing surfaces 623. The wiping edges 622 may be used for wiping and scrapping the capsular bag to detach and remove material, such as emulsified cataracts and/or lens and cortical material, from the capsular bag. The one or more polishing surfaces 623 are configured to polish the capsular bag. The cleaning member 600 may also include a textured material 630 (e.g., bristles, brushes, etc.) to clean and/or polish the capsular bag. The cleaning member 600 may also include one or more raised portions 632 for cleaning and/or polishing of the capsular bag.

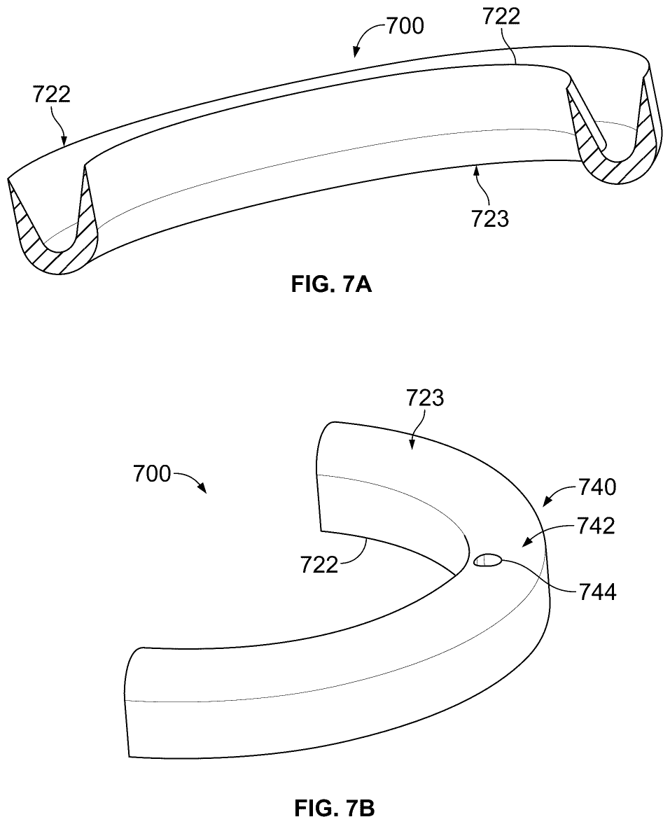

FIGS. 7A and 7B show another example of a cleaning member 700 for coupling with a handle or handpiece of a surgical instrument. For example, the cleaning member 700 may be configured to be coupled with the shaft 110 of the surgical instrument 100 of FIG. 4. The cleaning member 700 may be coupled with the handle or handpiece by any suitable means and in any suitable configuration. In some examples, the cleaning member 700 may include an attachment portion 740. The attachment portion 740 may be coupled with or attached to a handle or handpiece of a surgical instrument. The attachment portion 740 may be defined at any suitable location of the cleaning member 700. A connection mechanism 742 may couple the cleaning member 700 with a handle or handpiece of a surgical instrument. As shown, the connection mechanism 742 may comprise a through-hole 744 for receiving a connecting member (not shown), but any suitable mechanism may be used to couple the cleaning member 700 with a surgical instrument. Although the cleaning member 700 is shown as a separate component, it will be recognized that the cleaning member 700 may be integrated or combined with a handle or handpiece assembly to form a unitary or single piece surgical polishing instrument.

The cleaning member 700 may be used by a surgeon to polish a capsular bag of an eye. For example, the cleaning member 700 may be configured to detach and remove material (e.g., cortical material) from the capsular bag by wiping, scraping, detaching, etc. the material from the interior surfaces of the capsular bag. The cleaning member 700 may have a substantially curved cross-sectional shape. The cleaning member 700 may be constructed from a soft polymeric segment or any other suitable material.

As shown FIGS. 7A and 7B, the cleaning member 700 may include one or more polishing elements. The polishing element may be configured to polish the capsular bag. The polishing elements may include one or more wiping edges 722 and one or more polishing surfaces 723. The wiping edges 722 and the polishing surfaces 723 may be used for wiping, scrapping, and cleaning surfaces of the capsular bag to detach and remove material, such as emulsified cataracts and/or lens and cortical material, from the capsular bag. The cleaning member 700 may also include textured materials, bristles, brushes, raised portions, etc. for cleaning and/or polishing the capsular bag.

FIG. 8 illustrates a partial view of a surgical tool 800, in accordance with another example. The surgical tool is substantially similar to the surgical instrument 100 of FIG. 1 except for the direction of the curve of the cleaning member 812. For example, the curve of the cleaning member 812 may be positioned in the opposite direction as the cleaning member 112 of the surgical instrument 100 of FIG. 4. Although the cleaning member 812 is shown as a separate component, it will be recognized that the cleaning member 812 may be integrated or combined with a handle or handpiece assembly to form a unitary or single piece surgical polishing instrument.

This description is given for purposes of illustration and explanation. It will be apparent to those skilled in the relevant art that changes and modifications may be made to the invention described above without departing from its scope or spirit. For example, it will be recognized by those skilled in the art that the present invention may be combined with other handpieces. Further, the surgical instrument is not limited to phacoemulsification procedures, but may include any number of other surgical or therapeutic applications.

What is claimed is:

1. A surgical instrument for phacoemulsification procedures comprising:
a handpiece assembly; and
a cleaning member coupled with the handpiece assembly, the cleaning member configured to be inserted into an anterior chamber of an eye of a patient and into a capsular bag of the eye, wherein the cleaning member includes one or more polishing elements configured to detach material from the capsular bag.

2. The surgical instrument of claim 1, wherein the one or more polishing elements include one or more wiping edges configured for wiping a surface of the capsular bag, one or more polishing surfaces configured for polishing the capsular bag, or a combination thereof.

3. The surgical instrument of claim 1, wherein the handpiece assembly includes a handpiece portion removably attached to a grip portion.

4. The surgical instrument of claim 1, wherein the handpiece assembly comprises a shaft, and wherein the shaft is coupled with the cleaning member.

5. The surgical instrument of claim 1, wherein the cleaning member has a curvilinear or arc shape.

6. The surgical instrument of claim 2, further comprising a groove disposed between a first wiping edge and a second wiping edge of the one or more wiping edges.

7. The surgical instrument of claim 2, wherein at least of the one or one or more polishing surfaces is substantially smooth for polishing the capsular bag.

8. The surgical instrument of claim 2, wherein the one or more polishing surfaces includes a textured material or brush configured to detach material from surfaces of the capsular bag.

9. The surgical instrument of claim 2, wherein the one or more polishing surfaces includes a generally rounded portion having sufficient surface area configured for polishing the capsular bag.

10. The surgical instrument of claim 2, wherein the one or more polishing surfaces includes a first polishing surface configured for polishing the capsular bag and a second polishing surface for polishing the capsular bag.

11. The surgical instrument of claim 10, further comprising a groove disposed between the first polishing surface and the second polishing surface.

12. The surgical instrument of claim 10, wherein the first polishing surface and the second polishing surface form a V-shaped pattern.

13. The surgical instrument of claim 1, wherein the cleaning member is fabricated from a soft material.

14. A method of polishing a capsular bag of an eye of a patient comprising:
performing a phacoemulsification procedure on the eye of the patient;
inserting a surgical instrument having a cleaning member into the capsular bag of the patient, wherein the cleaning member includes one or more polishing elements configured to detach material from the capsular bag;
moving the cleaning member to cause the one or more polishing elements to detach the material from the capsular bag; and
removing the surgical instrument from the capsular bag.

15. The method of claim 14, wherein the one or more polishing elements include one or more wiping edges configured for wiping a surface of the capsular bag, one or more polishing surfaces configured for polishing the capsular bag, or a combination thereof.

16. The method of claim 14, wherein the cleaning member has a curvilinear or arc shape.

17. The method of claim 14, comprising a groove disposed between a first wiping edge and a wiping second edge of the one or more wiping edges.

18. The method of claim 15, wherein the one or more polishing surfaces includes a textured material configured to detach material from a surface of the capsular bag, and wherein the one or more polishing surface are substantially smooth for polishing the capsular bag.

19. The method of claim 15, wherein the one or more of the polishing surfaces includes a first polishing surface configured for polishing the capsular bag and a second polishing surface configured for polishing the capsular bag, and further comprising a groove disposed between the first polishing surface and the second polishing surface.

20. The method of claim 19, wherein the first polishing surface and the second polishing surface form a V-shaped pattern.
